(19) United States
(12) Patent Application Publication
(10) Pub. No.: US 2019/0269616 A1
(43) Pub. Date: Sep. 5, 2019

(54) STERILE PARENTERAL SUSPENSIONS

(71) Applicant: Emcure Pharmaceuticals Limited, Bhosari, Pune (IN)

(72) Inventors: Sougata Pramanick, Bhosari (IN); Aasiya Burhan, Bhosari (IN); Mukund Gurjar, Bhosari (IN)

(73) Assignee: Emcure Pharmaceuticals Limited, Bhosari, Pune (IN)

(21) Appl. No.: 15/780,198

(22) PCT Filed: Dec. 5, 2016
(86) PCT No.: PCT/IB2016/057341

§ 371 Date: May 30, 2018

(30) Foreign Application Priority Data
Dec. 7, 2015 (IN) 4617/MUM/2015

(51) Int. Cl. A61K 9/10; A61K 31/57; A61K 31/58; A61K 47/38; A61K 9/00

(52) U.S. Cl. CPC A61K 9/10; A61K 31/57; A61K 45/06; A61K 47/38; A61K 9/0019; A61K 31/58

(57) ABSTRACT

The present invention relates to bulk sterilized parenteral suspension formulations comprising water insoluble drugs suitable for parenteral use. Further, a process of preparation of such bulk sterilized suspension compositions employing conventional sterilization process under homogenization is also disclosed. The sterilization under homogenization is carried for prolonged period and the finally aseptically filled into suitable container closure systems. The bulk sterilized suspensions prepared by using the current invention exhibited good physical and chemical stability.

This application is the U.S. National Stage filing of International Patent Application Number PCT/IB2016/057341 filed on Dec. 5, 2016 which claims the benefit of Indian Provisional Application No. 4617/MUM/2015, filed on 7 Dec. 2015, which are hereby incorporated by reference in their entirety.

### FIELD OF THE INVENTION

The present invention relates to galenic preparations, particularly to pharmaceutical aqueous suspension composition of drugs suitable for parenteral administration. The present invention also relates to bulk sterilized suspension compositions and a process for preparation of such pharmaceutical parenteral suspension composition.

### BACKGROUND OF THE INVENTION

With innovations in new target and/or disease specific drug development, majority of the new chemical entities coming out of drug discovery groups at pharmaceutical companies have been faced with poor water solubility challenges. Further, majority of the water insoluble drugs are found in oncology, anti-infective, central nervous system and anti-viral therapeutic indications. With the increase in patient population in these diseases, there has been a strong interest in suspension dosage forms for injectable applications.

Injectable suspension formulations are administered by intravenous (IV), subcutaneous (SC) or intramuscular (IM) routes. Parenteral suspensions are heterogeneous systems that typically consist of a solid phase dispersed in a liquid phase, the liquid phase being aqueous or nonaqueous. To be effective and pharmaceutically acceptable, injectable suspensions should preferably be sterile, stable, resuspendable, syringeable, injectable, isotonic and nonirritating. The foregoing characteristics result in manufacturing, storage, and usage requirements that make injectable suspensions one of the most difficult dosage forms to develop.

The need for sterility in these parenteral suspension formulations is mandated both by concerns for the general public safety, and a pharmaceutical company's reputation for producing quality products. Formulations intended for the parenteral administration are mandated by the health authorities all over the world to be sterile, even though after the first access to what is intended to be a multi-use package, they tend to loose their sterility for subsequent uses. Therefore, selection of an ideal sterilization method is quite cumbersome for parenteral suspension products.

Terminal sterilization of the final product by autoclaving could cause physical or chemical instability—the increase or decrease in solubility of the suspended drug particles, and destroy the integrity of the suspension. Further terminal sterilization of a suspension formulation may alter the viscosity of the product affecting suspending ability of the vehicle. Technique of gamma radiation may pose the problem of formation of free radicals by ionizing radiation. Filtration, another method of sterilization, physically removes microorganisms using bacteria-retentive filters, using an exclusion principle based on size. However, if the particle size of the parenteral injectable suspension product is small enough, then only filtration can be employed. Therefore, normally, aseptic manufacturing process is employed for the parenteral suspension product, wherein it is necessary to sterilize separately the individual component of the suspension formulation before combining them.

Published literature also suggests the criticality of sterilization method for the parenteral suspension formulations. U.S. Pat. No. 3,962,430 discloses sterilization of solid non-electrolyte medicinal agents which contains excess sodium chloride by heating in an aqueous suspension by autoclaving at 121° C. for 20-30 minutes at same temperature in final sealed containers.

U.S. Pat. No. 4,029,782 discloses storage stable pharmaceutical suspension of Cefazolin for parenteral administration in which aqueous solution of excipients is autoclaved for 60 min at 115° to 125° C. and then drug is aseptically added under agitation to form stable storage suspension.

U.S. Pat. No. 6,066,292 discloses sterilization process for pharmaceutical suspensions that involves heat-sterilizing an aqueous solution of a viscosity enhancer, to result in a first sterile pre-mix, sterile-filtering an aqueous solution of a mixture of a pharmaceutically-active compound, which results in a second sterile pre-mix, heat-sterilizing a mixture of water, a Water-insoluble pharmaceutical to form third pre-mix; combining all three pre-mixes in sterile fashion to achieve a sterile suspended pharmaceutical formulation.

U.S. Pat. No. 6,495,534 discloses preparation of stable aqueous suspensions of medroxyprogesterone acetate that utilizes steam sterilization of aqueous solution of excipients & then dispersion of drug further homogenization and aseptic filling into final containers.

U.S. Pat. No. 7,892,483 discloses process for the sterilization of a steroid that involves heat treating the steroid in the form of a wet mass consisting essentially of the steroid, water and surfactant.

PCT Application No. WO1999061001A1 discloses compositions of submicron- to micron-sized particles of water-insoluble biologically active substances that are stabilized by thermoprotecting agents, can be terminally steam sterilized without any significant increase of mean particle size.

Thus, there exists a need for more flexible methods of sterilizing parenteral suspension formulations having one or more water insoluble components.

### SUMMARY OF THE INVENTION

The present invention is directed to a bulk sterilized injectable suspension composition comprising water insoluble drug and pharmaceutically acceptable excipients. Such bulk sterilized composition is then dispensed into suitable container closure systems.

The present invention further provides a process for preparation of such bulk sterilized suspension and unit package formulation of the same.

In the preferred embodiment, the bulk sterilized injectable suspension composition is prepared by using bulk sterilization process using conventional sterilization methods.

In another preferred embodiment, the bulk sterilized injectable suspension composition is prepared by using moist heat method.

In another preferred embodiment, the bulk sterilized injectable suspension composition is prepared by using conventional methods under homogenization.

In another preferred embodiment, the bulk sterilized injectable suspension composition is prepared by using moist heat under homogenization.

In another preferred embodiment, the bulk sterilized injectable suspension composition is prepared by using moist heat for prolonged period.

In another preferred embodiment, the bulk sterilized injectable suspension composition is prepared by using moist heat for prolonged period under homogenization.

### DETAILED DESCRIPTION OF THE INVENTION

The present invention is directed to an injection suspension composition. Further the present invention is directed to a bulk sterilized injectable suspension composition comprising water insoluble drug. Typically, the bulk sterilization process involves preparation of uniform suspension and then bulk sterilizing the suspension formulation under homogenization to yield sterile bulk suspension.

Typically, the moist or dry heat methods are not at all preferred for sterilizing injectable suspension compositions as heat affects the homogeneity or uniformity of the final product either by catalyzing the formation of loose agglomerations called “curds,” or, if the curds become compacted and fuse, “cakes” of suspended particles. Curds and cakes negatively impact on the patient or caregiver's ability to re-suspend the product easily and provide uniform dosing. But the present inventors have unexpectedly discovered that any sterilization process could be employed if sterilization is carried out at bulk solution stage for preparing parenteral suspension of water insoluble or poorly soluble drugs. Stable suspension retaining the desired physical and chemical attributes of the non-sterile product could be obtained if the bulk sterilization process is carried out under homogenization.

The key step in the sterilization process is bulk sterilization of homogenous suspension formulation using conventional methods such as dry heat or moist heat. Further, preferably bulk sterilization of homogenous suspension formulation is carried out for prolonged period than conventional process. Continued thermal exposure during extended autoclaving results in maturing of suspension due to controlled flocculation. Most commonly, controlled flocculation is achieved by adding flocculating agents, like electrolytes, polymers or surfactant to stabilize the suspension. Once the final particle size is achieved, no further changes occur in the particles interactions. This causes physicochemical parameters (like pH, particle size, zeta potential, viscosity, drug release profile etc) to become constant over time. In the present invention along with flocculating agents, thermal exposure for prolonged period of time during bulk sterilization causes the same effect and results in a stable suspension with stable physicochemical properties.

Further, the homogenous bulk sterilized suspension is preferably achieved by homogenization. The suspension formulation is kept as a homogenized mixture throughout the sterilization process, which leads to uniform dispersion of suspended particles. This avoids the formation of hard cakes, which are difficult to redisperse that are usually observed in traditional autoclaving processes. Without intending to be bound by any theory, the applicants believe that the sterilization process with simultaneous homogenization for prolonged period results in stable suspension such that there are no trends during shelf life storage. The extended period of up to 2 hours or preferably about 60 min autoclaving is therefore preferable.

The formulations prepared using the present invention also exhibited good stability throughout shelf life as the impurities observed were well below the specified limits.

The drugs referred to in the present invention relates to the pharmaceutically active agents suitable for parenteral use and or normally supplied as suspension formulation such as corticosteroids, steroids and non-steroid drugs selected from the group consisting of cortisone, cortisone acetate, dexamethasone, dexamethasone acetate, dexamethasone tertiary butyl acetate, hydrocortisone acetate, prednisolone acetate, betamethasone acetate and methyl prednisolone, steroids selected from the group consisting of betamethasone, fluticasone (e. g., as propionate), budesonide, tipredane, dexamethasone, beclomethasone (e.g., as diproprionate), prednisolone, flucinolone, triamcinolone (e. g., as acetonide), mometasone (e.g., as furoate), rofleponide (e. g., as palmitate), flumethasone, flunisolide, ciclesonide, deflazacort, cortivazol, 16a,17a-butylidenedioxy-6a,9a-difluro-11 ss, 21-dihydroxy-pregna-1, 4-diene3,20-dione; 6a,9a-difluro-11 ss-hydroxy-16a,17a-butylidenedioxy-17ss-methylthio-androsta-4-ene-3-one; 16a, 17a-butylidenedioxy-6a,9a-difluoro-11ss-dydroxy-3-oxo-androsta-1,4-diene-17p-carbothioic acide S-methyl ester; methyl 1 9a-chloro-6a-fluoro-1 1 ss-hydroxy-16oc-methyl-3-oxo-17a-propionyloxy-androsta-1,4-diene-17a-carboxylate; 6a, 9a-difluoro-11 ss-hydroxy-16a-methyl-3-oxo-17a-propionyloxy-androsta-1,4-diene-17p-carbothioic acid-(2-oxo-tetrahydrofuran-3-yl) ester, androsterone, 17-methyltestosterone, testosterone, testosterone borate, dienstrol, diethylstilbesterol, estradiol, estradiol-17-acetate, estradiol benzoate, medroxyprogesterone acetate and ethinyl estradiol-3-methyl ether and non-steroids selected from the group consisting of propylidone, primidone, diphenylhydantoin, indomethacin, paliperidone, risperidone, NSAID drugs such as diclofenac and its salts, antibiotics such as cephalosporins, ciprofloxacin and thiabendazole medicinals. The only criterion for selection of the active ingredient is that it should be available as parenteral suspension product.

Typically, the present invention also relates to preparation of sterile parenteral suspension of drugs with other pharmaceutically acceptable excipients. The compositions include drugs suitable for parenteral administration and pharmaceutically acceptable excipients selected from the group consisting of suspending agents and/or wetting agents, viscosity enhancers, preservatives, surfactants, buffering agents and/or pH adjusting agents and vehicle.

The term “wetting agents” or “surfactants”, as used herein, refers to another essential ingredient useful for suspension of this invention. If suspension is provided in a water vehicle, an essentially water soluble surfactant is required. Listed among the pharmaceutically acceptable surfactants which have been found to be useful in suspension of the present invention are polyoxyethylene (20) sorbitan monolaurate (polysorbate 20), polyoxyethylene (20) sorbitan monostearate (polysorbate 60) polyoxyethylene (20) sorbitan monooleate (polysorbate 80), polyoxyl 40 stearate, polyoxyethylene 50 stearate, sodium lauryl sulfate, and the like. The surfactants can be employed in concentration from about 0.1 to about 1.0 percent (W/V) of the suspension. Moreover, the presence of the surfactant aids in establishing zeta forces surrounding such particles which tend to discourage agglomeration of the particles and retard settling of the suspension. It will be understood by those skilled in the art that optimum quantities of surfactants are important to prepare stable suspensions, but that extra amounts are of no particular use although no mischief results there from. Consequently, excess amounts of surfactants are of no particular benefit and do add to the cost of preparing the suspension.

The term “viscosity adjusting agents” or “suspending agents”, as used herein, refers to agents that are needed to provide a suspension in which the settling of the particles is impeded and at the same time remains sufficiently fluid to be syringeable. By syringeable it is meant that the suspension can be withdrawn easily from an ampoule into a 5 cc syringe with a 20 gauge needle and subsequently injected from such a syringe through the 20 gauge needle into muscle tissue.

Viscosity adjusting agents which are useful in the present invention include povidone, sodium carboxymethylcellulose, polyvinylpyrrolidone compounds and polyethylene glycols. Preferred examples of polyethylene glycols are those having a molecular weight from about 300 to about 6000, e.g. polyethylene glycol 3350 and polyethylene glycol 4000. Preferred polyvinylpyrrolidone (PVP) compounds according to the invention are those having a molecular weight from about 7000 to about 54000, for instance PVP K12, K17, K25 and K30, in particular K12 and K17, PVP K17 being the most preferred. Other suitable thickening or viscosity agents are for instance well known cellulose derivatives (e.g. methylcellulose, carboxymethylcellulose, hydroxyethylcellulose and hydroxypropylmethylcellulose), gelatin and acacia, in particular methylcellulose.

The term “preservatives”, as used herein, refers to agents such as phenol, chlorobutanol, benzyl alcohol, methylparaben, propylparaben, benzalkonium chloride and cetylpyridinium chloride. In addition, the formulations of the present invention may also include tonicity-adjusting agents. Suitable tonicity adjusting agents are for instance sodium chloride, sodium sulfate, dextrose, mannitol and glycerol.

The term “buffering agents” or “pH adjusting agents”, as used herein, refers to agents such as acetate, citrate, tartrate, phosphate, Triethanolamine (TRIS), sodium hydroxide, hydrochloric acid etc.

In addition, the formulations of the present invention may also include metal chelating agents, antioxidants or thiol-containing compounds and preservatives. Suitable metal chelating agents are for instance ethylenediamine-tetracetic acid salts (e.g. edetate di sodium). Suitable antioxidants are for instance ascorbic acid derivatives (e.g. ascorbic acid, erythorbic acid, sodium ascorbate), thiol derivatives (e.g. thioglycerol, cysteine, acetylcysteine, cystine, dithioerythreitol, dithiothreitol, gluthathione), tocopherols, butylated hydroxyani sole, butylated hydroxytoluene, sulfurous acid salts (e.g. sodium sulfate, sodium bisulfate, acetone sodium bisulfate, sodium metabisulfite, sodium sulphite, sodium formaldehyde sulfoxylate, sodium thiosulfate) and nordihydroguaiareticacid.

The vehicle utilized can be aqueous or non-aqueous vehicle or mixture of aqueous and non-aqueous vehicles could also be used.

The bulk sterilized parenteral suspension composition and process of preparation according to the present invention typical involves the following steps:
a) Preparing an aqueous solution of suitable excipients
b) Adding water insoluble drug to the solution of step a,
c) Mixing and homogenizing the suspension of step b
d) Bulk sterilizing the suspension of step c using conventional sterilization method for about 15 min to 2 hours.
e) Aseptically filling the bulk sterilized suspension in to the final containers.

The suitable blenders and/or homogenizers used for mixing can be planetary motion mixers, sigma blenders, colloid mill, over head stirrers, emulsifiers, homogenizers, high pressure homogenizers etc. Further the solution can be filtered through PVDF filter.

The bulk sterilization of the prepared suspension can be performed using conventional methods such as dry heat, moist heat methods, more preferably moist heat process using conventional procedure for example by autoclaving at 121° C. for 15 min. However, the bulk sterilization of the suspension could also be carried out for extended period of time e.g. for about 15 min, for about 30 min, about 45 min, about 60 min, about 80 min or about 120 min or longer.

The bulk sterilized suspension prepared by the above process is aseptically filled into final containers. The containers used for the purpose can be selected from vials, ampoules, plastic containers, pre-filled syringe systems etc. The filled containers are stoppered using suitable closure systems used for parenteral formulations.

Typically, a suspension formulation of Triamcinolone Acetonide, Medroxyprogesterone acetate is prepared by using the invention described herein.
a) Prepare an aqueous solution of preservative, surfactant and/or wetting agent, viscosity adjusting agents and tonicity adjusting agent,
b) The pH of the solution of step (a) is adjusted with suitable pH adjusting agents,
c) The above solution is filtered through PVDF filter,
d) Add water insoluble drug to the solution of step (c),
e) Mixing the suspension of step (d) to form uniform suspension,
f) The volume of the suspension is finally made up with water for injection,
g) Bulk sterilizing the suspension of step (f) using moist heat,
h) Aseptically filling the bulk sterilized suspension in to the final containers.

Typically the autoclaving for preparation of bulk sterilized suspension employed is for less than about 2 hour, preferably for about 15 min to 90 min. Such formulation shows not more than about 2% of total impurities when stored at pharmaceutically acceptable conditions. Further the each individual impurity is not more than about 0.3% that qualifies the acceptance criteria.

There is typically no change in the particle size observed for formulation prepared by employing the process of present invention before and after sterilization. When the initial particle size is very small, variation of up to 30% is observed when sterilized according to present process.

The invention is further explained with the help of following illustrative examples, however, in no way these examples should be construed as limiting the scope of the invention.

### EXAMPLES

### Example 1: Triamcinolone Acetonide Injectable Suspension USP

TABLE 1
Composition of Triamcinolone parenteral suspension

| | | Formulation I | |
|---|---|---|---|
| Sr. No | Ingredients | 40 mg/mL | 10 mg/mL |
| 1 | Triamcinolone Acetonide | | |
| | USP | | |
| 2 | Benzyl Alcohol NF | 9.0 | 9.0 |
| 3 | Polysorbate 80 NF | 0.4 | 0.4 |
| 4 | Carboxymethylcellulose, | 7.5 | 7.5 |
| | Sodium USP | | |
| 5 | Sodium Chloride USP | 6.5 | 6.5 |
| 6 | Sodium Hydroxide NF | q.s. to adjust pH | q.s. to adjust pH |
| 7 | Hydrochloric acid NF | q.s. to adjust pH | q.s. to adjust pH |
| 8 | Water for Injection USP | q.s. to 1 mL | q.s. to 1 mL |

The formulation of Triamcinolone acetonide parenteral suspension is prepared in accordance with the present invention as detailed below:

Aqueous phase of suspension is prepared by dissolving Polysorbate 80 in WFI and further adding benzyl alcohol, carboxymethylcellulose sodium and sodium chloride one after other until complete dissolution of the added excipients occur. The solution is then filtered through PVDF filter. To the filtered solution drug is added. The dispersion is subjected to stirring for 20-30 minutes to get uniform mixture. The volume is made up and the suspension is homogenized. Further the suspension is bulk sterilized at 121° C. for 15-60 minutes under homogenization. The final sterilized suspension is aseptically transferred into final containers. The sterilized formulation is then evaluated for various parameters and results are summarized in Table-2.

TABLE 2
Analytical results of bulk sterilization

| | | | Batch 1 | Batch 2 | Batch 3 | Batch 4 |
|---|---|---|---|---|---|---|
| | | | 15 min | 30 min | 45 min | 60 min |
| Test | Specification | Non sterile | Sterilization | Sterilization | Sterilization | Sterilization |
| Description | White to off- | White to off-white colored suspension | | | | |
| | white colored | | | | | |
| | suspension | | | | | |
| pH | Between 5.0 and | 5.8 | 5.8 | 5.7 | 5.7 | 5.7 |
| | 7.5 | | | | | |
| Assay | NLT 90.0% and | 99.4 | 101.1 | 101.1 | 101.4 | 101.4 |
| (By HPLC) | NMT 115.0% | | | | | |
| Assay of | NLT 80.0% & | 96.2 | Not scheduled | | | 95.7 |
| Benzyl | NMT 120.0% | | | | | |
| alcohol | | | | | | |
| Particle | d (90) | 9.9 | 11.6 | 12.2 | 11.3 | 14.7 |
| size | | | | | | |
| Viscosity | — | 34 | 19 | 18 | 18 | 16 |
| (cP) | | | | | | |
| Zeta | — | −48.4 | −42.1 | −41.1 | −46.7 | −44.3 |
| potential | | | | | | |
| (mV) | | | | | | |

The impurity profile was also evaluated for the sterilized products and results are summarized in Table 3:

TABLE 3
Stability Profile of Bulk Sterilized Suspension Formulation

| | | | Invention | |
|---|---|---|---|---|
| Specification | | | Non sterile | Batch 4 |
| Related | 1,2 dihydro | NMT 0.3% | 0.0 | 0.0 |
| Compounds | triamcinolone | | | |
| by HPLC | acetonide | | | |
| | Triamcinolone | NMT 0.3% | BLQ | BLQ |
| | acetonide 21- | | | |
| | hydrated aldehyde | | | |
| | Any unspecified | NMT 0.3% | 0.1 | 0.1 |
| | impurity | | | |
| | Total impurities | NMT 2.0% | 0.4 | 0.3 |

It is observed from the above results that the bulk sterilized Triamcinolone acetonide suspension formulation prepared in accordance to the present invention exhibited impurity profile within the specified limit i.e., each of the impurity is not more than 0.3% and the total impurities are not more than 2.0%.

### Example 2: Medroxyprogesterone Acetate Injectable Suspension USP (150 mg/mL)

TABLE 4
Composition of Medroxyprogesterone parenteral suspension

| | | Formulation II |
|---|---|---|
| S. No. | Ingredients | (mg/mL) |
| 1 | Medroxyprogesterone Acetate USP | 150 mg |
| 2 | Polyethylene glycol 3350 | 28.9 mg |
| 3 | Polysorbate 80 | 2.41 mg |
| 4 | Sodium chloride | 8.68 mg |
| 5 | Methylparaben | 1.37 mg |
| 6 | Propylparaben | 0.150 mg |
| 7 | Sodium Hydroxide NF | q.s. to adjust pH |
| 8 | Hydrochloric acid NF | q.s. to adjust pH |
| 9 | Water for injection | quantity sufficient |

The sterile suspension of Medroxyprogesterone acetate is prepared as described in Example 1 and using excipients summarized in Table 4. Analytical results of the formulation are summarized in Table 5.

TABLE 5
Analytical results of Formulation II sterilization a 121° C. for 45 minutes

| | | | | Formulation II | | | |
|---|---|---|---|---|---|---|---|
| | | | | | Sterilization at 121° C. for | | |
| | | | | | 45 minutes | | |
| Sr. | | | | | 40° C./75% RH | | |
| No | Test | | Specification | Initial | 1 M | 2 M | 3 M |
| 1 | Description | | White to off-white | Complies | Complies | | |
| | | | suspension | | | | |
| 2 | pH | | 3-7 | 5.38 | 5.11 | 5.31 | 5.24 |
| 3 | PSD | | d (99) | 26 | 25 | 25 | 25 |
| 4 | % Assay | Medroxyprogesterone | NLT 90.0% & NMT | 104.8 | 101.2 | 101.4 | 102.7 |
| | | Acetate | 110.0% | | | | |

| Related Substances | | | | | |
|---|---|---|---|---|---|
| Impurity A | NMT 0.2% | 0.04 | 0.04 | 0.04 | 0.03 |
| Impurity B | NMT 0.2% | 0.13 | 0.12 | 0.14 | 0.14 |
| Impurity C | NMT 0.2% | 0.03 | 0.02 | 0.03 | ND |
| Impurity D | NMT 0.2% | 0.05 | 0.04 | 0.04 | 0.02 |
| Impurity E | NMT 0.2% | 0.06 | 0.01 | 0.04 | 0.01 |
| Impurity F | NMT 0.2% | 0.14 | 0.19 | 0.15 | 0.18 |
| Impurity G | NMT 0.2% | ND | ND | ND | ND |
| Impurity H | NMT 0.2% | ND | ND | ND | ND |
| Impurity I | NMT 0.2% | 0.17 | 0.15 | 0.17 | 0.18 |
| Unknown max. | NMT 0.2% | 0.04 | 0.01 | 0.01 | 0.01 |
| Total impurities | NMT 1.0% | 0.6 | 0.46 | 0.60 | 0.41 |

It is observed from the above results that the bulk sterilized Medroxyprogesterone acetate suspension formulation prepared in accordance to the present invention exhibited impurity profile within the specified limit i.e., each of the impurity is not more than 0.2% and the total impurities are not more than 1.0%.

1. A bulk sterilized injectable suspension composition comprising water insoluble drug and pharmaceutically acceptable excipients.

2. The bulk sterilized injectable suspension composition as claimed in claim 1, wherein the composition is bulk sterilizes using conventional methods.

3. The bulk sterilized injectable suspension composition as claimed in claim 2, wherein the bulk sterilization process is performed under homogenization.

4. The bulk sterilized injectable suspensions composition as claimed in claim 3, wherein the bulk sterilization process is performed using moist heat under homogenization.

5. The bulk sterilized injectable suspensions composition as claimed in claim 1, wherein the bulk sterilization process is performed for prolonged period of time.

6. The bulk sterilized injectable suspensions composition as claimed in claim 6, wherein the bulk sterilization process is performed under homogenization.

7. The bulk sterilized injectable suspensions composition as claimed in claim 6, wherein the bulk sterilization process is carried out for a period of 15 min to 2 hours under homogenization.

8. The bulk sterilized injectable suspensions composition as claimed in claim 8, wherein the bulk sterilization process is carried out for a period of about 1 hour under homogenization.

9. The bulk sterilized injectable suspension composition as claimed in claim 1, wherein the water insoluble drugs are pharmaceutically active agents suitable for parenteral use and are selected from the group comprising of cortisone, dexamethasone, hydrocortisone, prednisolone, betamethasone (e.g., as acetate, dipropionate), dexamethasone tertiary butyl acetate and methyl prednisolone, steroids selected from the group consisting of fluticasone (e. g., as propionate), budesonide, tipredane, flucinolone, triamcinolone (e. g., as acetonide), mometasone (e.g., as furoate), rofleponide (e. g., as palmitate), flumethasone, flunisolide, ciclesonide, deflazacort, cortivazol, 16a,17a-butylidenedioxy-6a,9a-difluro-11 ss, 21-dihydroxy-pregna-1, 4-diene3,20-dione; 6a,9a-difluro-11 ss-hydroxy-16a, 17a-butylidenedioxy-17ss-methylthio-androsta-4-ene-3-one; 16a, 17a-butylidenedioxy-6a,9a-difluoro-1 1 ss-dydroxy-3-oxo-androsta-1,4-diene-17p-carbothioic acide S-methyl ester; methyl 1 9a-chloro-6a-fluoro-1 1 ss-hydroxy-16oc-methyl-3-oxo-17a-propionyloxy-androsta-1,4-diene-17a-carboxylate; 6a,9a-difluoro-11 ss-hydroxy-16a-methyl-3-oxo-17a-propionyloxy-androsta-1,4-diene-17p-carbothioic acid-(2-oxo-tetrahydrofuran-3-yl) ester, androsterone, 17-methyltestosterone, testosterone, testosterone borate, dienstrol, diethylstilbesterol, estradiol, estradiol-17-acetate, estradiol benzoate, medroxyprogesterone acetate and ethinyl estradiol-3-methyl ether and non-steroids selected from the group consisting of propylidone, primidone, diphenylhydantoin, indomethacin, paliperidone, risperidone, NSAID drugs and their salts or esters, antibiotics such as cephalosporins, ciprofloxacin and thiabendazole drugs.

10. The process for preparation of bulk sterilized injectable suspensions comprising water insoluble drug and pharmaceutically acceptable excipients comprising the following steps:
a) Prepare an aqueous solution of suitable excipients
b) Add water insoluble drug to the solution of step (a),
c) Mixing and homogenizing the suspension of step (b)
d) Bulk sterilizing the suspension of step c using conventional sterilization method for about 15 min to 2 hours.
e) Aseptically filling the bulk sterilized suspension in to the final containers.
